(19) United States
(12) Patent Application Publication
(10) Pub. No.: US 2024/0325325 A1
(43) Pub. Date: Oct. 3, 2024

(54) LIPOSOME ENCAPSULATING CYSTEAMINE AND RESVERATROL IN SEPARATE COMPARTMENTS

(71) Applicant: KING FAISAL UNIVERSITY, AL-AHSA (SA)

(72) Inventors: Sree Harsha NAGARAJA, AL-AHSA (SA); Anroop B. NAIR, AL-AHSA (SA); Mahesh ATTIMARAD, AL-AHSA (SA); Katharigatta N. VENUGOPALA, AL-AHSA (SA); Bander E. AL-DHUBIAB, AL-AHSA (SA); Santosh FATTEPUR, AL-AHSA (SA); Teeka Sathiesh ROOPASHREE, AL-AHSA (SA); Arshia SHARIFF, AL-AHSA (SA); Mohammed F. ALDAWSARI, AL-AHSA (SA); Sibghatullah SANGI, AL-AHSA (SA)

(21) Appl. No.: 18/127,862

(22) Filed: Mar. 29, 2023

(51) Int. Cl. A61K 31/145; A61K 9/127; A61K 31/05

(52) U.S. Cl. CPC A61K 31/145; A61K 9/127; A61K 31/05

(57) ABSTRACT

The liposome encapsulating cysteamine and resveratrol in separate compartments is a liposome having a lipid or lipid bilayer shell and a hydrophilic core. The hydrophilic core contains a hydrophilic antioxidant, which in some embodiments, is cysteamine or pharmaceutically acceptable salts thereof, such as hydrochlorides or bitartrates. The region surrounding the hydrophilic core contains lipids or phospholipids and at least one lipophilic antioxidant, such as resveratrol. The lipophilic contents of the liposome surrounding the hydrophilic core tend to partition from the hydrophilic core and associate with the lipid or lipid bilayer shell. In some embodiments, the liposome composition may be used for the treatment of acute lung injury or acute respiratory distress syndrome, the antioxidants helping to reduce or alleviate inflammation, whether resulting from disease or trauma. The liposomal composition may be administered in a pharmaceutically acceptable carrier by intravenous, endotracheal, intraperitoneal, or other routes of administration.

### BACKGROUND

### 1. Field

The disclosure of the present patent application relates to drug delivery for the treatment of pulmonary infections, and particularly to a liposome encapsulating cysteamine and resveratrol in separate compartments, the composition is used for treatment of acute respiratory distress syndrome (ARDS) and inflammation of the lungs.

### 2. Description of the Related Art

Lung inflammation in the form of acute lung injury (ALI) or acute respiratory distress syndrome (ARDS) is the clinical syndrome of respiratory failure with considerably high morbidity and mortality. Even if the patient survives through ALI, their long-term quality of life is adversely affected. Recent advances have been made in the understanding of the epidemiology, pathogenesis, and treatment of this disease. However, more progress is needed to further reduce mortality and morbidity from ALI and ARDS. Because lung inflammation leading to respiratory failure is so common, both in the United States and worldwide, one can say that ALI/ARDS is an unmet medical need. In other words, novel therapies need to be developed to further improve clinical outcomes. In such a scenario, the development of new compositions and treatment regimens for the management of lung inflammation that provides synergistic effects and minimal toxicity to the subject being treated would be advantageous. Thus, a liposome encapsulating cysteamine and resveratrol in separate compartments solving the aforementioned problems is desired.

### SUMMARY

The liposome encapsulating cysteamine and resveratrol in separate compartments is a liposome having a lipid or lipid bilayer shell and a hydrophilic core. The hydrophilic core contains a hydrophilic antioxidant, which in some embodiments, is cysteamine or pharmaceutically acceptable salts thereof, such as hydrochlorides or bitartrates. The region surrounding the hydrophilic core contains lipids or phospholipids and at least one lipophilic antioxidant, such as resveratrol. The lipophilic contents of the liposome surrounding the hydrophilic core tend to partition from the hydrophilic core and associate with the lipid or lipid bilayer shell. In some embodiments, the liposome composition may be used for the treatment of acute lung injury or acute respiratory distress syndrome, the antioxidants helping to reduce or alleviate inflammation, whether resulting from disease or trauma. The liposomal composition may be administered in a pharmaceutically acceptable carrier by intravenous, endotracheal, intraperitoneal, or other routes of administration.

These and other features of the present subject matter will become readily apparent upon further review of the following specification and drawings.

### BRIEF DESCRIPTION OF THE DRAWINGS

FIG. 1 is a SEM photograph of liposomes encapsulating cysteamine and resveratrol in separate compartments of the composition Example 1.

FIG. 2 is a chart comparing cellular uptake of cysteamine and resveratrol from liposomal composition Example 1 and from solution formulation of equivalent concentration.

FIG. 3 is a chart showing the biocompatibility of liposomal formulation of the composition Example 1 in L929 cells.

FIG. 4 is a chart showing the in vitro lung deposition using Twin Stage Impinger depicting deposition of the liposomal composition of the composition Example 1 in different parts of the lung.

FIG. 5 is a chart showing the Anderson Cascade Impactor deposition pattern of the liposomal composition of Example 1 on various stages of impactor.

FIG. 6A is a chart comparing pulmonary hemorrhage in terms of the optical density of BALF (bronchioalveolar lavage fluid) from various groups namely, Normal Control, Disease Control, and liposomal composition of Example 1

FIG. 6B is a chart comparing Total protein levels in BALF from various groups namely, Normal Control, Disease Control, and liposomal composition of Example 1.

FIG. 6C is a chart comparing Neutrophil levels in BALF from various groups namely, Normal Control, Disease Control, and liposomal composition of Example 1.

FIG. 7A is a chart comparing the levels of lipid peroxidation (Thiobarbituric acid reactive substances; TBARS) in BALF from groups namely, Normal Control, Disease Control, and liposomal composition of Example 1 treatment group.

FIG. 7B is a chart comparing levels of inflammatory marker Interleukin-18 (IL-1B) in BALF from Normal Control, Disease Control, and liposomal Example 1 treatment group.

FIG. 7C is a chart comparing levels of inflammatory marker Interleukin-6 (IL-6) in BALF from Normal Control, Disease Control, and liposomal composition of Example 1 treatment group.

FIG. 7D is a chart comparing levels of inflammatory marker Tumore Necrosis Factor α (TNF-α) in BALF from Normal Control, Disease Control, and liposomal composition of Example 1 treatment group.

Similar reference characters denote corresponding features consistently throughout the attached drawings.

### DETAILED DESCRIPTION OF THE PREFERRED EMBODIMENTS

The liposome encapsulating cysteamine and resveratrol in separate compartments is a liposome having a lipid or lipid bilayer shell and a hydrophilic core. The hydrophilic core contains a hydrophilic antioxidant, which in some embodiments, is cysteamine or pharmaceutically acceptable salts thereof, such as hydrochlorides or bitartrates. The region surrounding the hydrophilic core contains lipids or phospholipids and at least one lipophilic antioxidant, such as resveratrol. The lipophilic contents of the liposome surrounding the hydrophilic core tend to partition from the hydrophilic core and associate with the lipid or lipid bilayer shell. In some embodiments, the liposome composition may be used for the treatment of acute lung injury or acute respiratory distress syndrome, the antioxidants helping to reduce or alleviate inflammation, whether resulting from disease or trauma. The liposomal composition may be administered in a pharmaceutically acceptable carrier by intravenous, endotracheal, intraperitoneal, or other routes of administration.

Various aspects now will be described more fully hereinafter. Such aspects may, however, be embodied in many different forms and should not be construed as limited to the embodiments set forth herein. Rather, these embodiments are provided so that this disclosure will be thorough and complete, and will fully convey its scope to those skilled in the art.

“Administering” or “administration” as used herein means the introduction of a foreign molecule into a cell or host. The term is intended to be synonymous with the term “delivery” or “delivering”. Suitable routes of administration, without limitation, are intravenous, intra-arterial, intramuscular, subcutaneous, intraperitoneal, intratracheal, infusion, oral, or topical.

As used herein, the phrase “antioxidant” is synonymous with “anti-inflammatory agent” and refers to compounds that prevent inflammation. Generally, antioxidant agents may reduce inflammation by: (1) scavenging reactive oxygen species (ROS) or free radical derivatives, such as singlet oxygen and hydrogen peroxide, and (2) by blocking major signaling pathways, such as NF-κB and mitogen-activated protein kinases that have the main role in the production of various proinflammatory mediators.

An amount of liposomal-resveratrol that yields a therapeutically-effective amount of resveratrol after administration is an amount of resveratrol that is effective to ameliorate or minimize the clinical impairment or symptoms of the inflammation, in either a single dose or in multiple doses.

As used herein, a “lung inflammation” or “lung injury” involves disruption of the lung endothelial and epithelial barriers. The alveolar-capillary membrane comprised of the microvascular endothelium, interstitium, and alveolar epithelium shows pathophysiological changes. Cellular characteristics of lung inflammation include loss of alveolar-capillary membrane integrity, excessive transepithelial neutrophil migration, and release of proinflammatory, cytotoxic mediators.

The term “patient” refers to an individual afflicted with a disease characterized by neoplasia. In particular, a patient (i.e., a host) is an animal (i.e., mammal) or human.

As used herein, “pharmaceutical formulations” include formulations for human and veterinary use with no significant adverse effect. “Pharmaceutically acceptable carrier,” as used herein, refers to a composition or formulation that allows for the effective distribution of the agents of the instant invention in the physical location most suitable for their desired activity, and “pharmaceutically acceptable carrier” refers to a buffer, stabilizer or other material well known to those skilled in the art. Such materials should be non-toxic and should not interfere with the efficacy of the active ingredient. The precise nature of the carrier or other material may depend on the route of administration.

As used herein “synergistic effect” or “therapeutic synergy” refers to a clinical observation wherein a combination of cysteamine and resveratrol administered to a subject via injection of liposomes with co-encapsulated cysteamine and resveratrol provides more than additive effect of resveratrol administered via a liposomal-resveratrol formulation alone and more than cysteamine administered via liposome-entrapped cysteamine.

Reference to a “therapeutically effective amount,” intends an amount of a compound sufficient to show benefit to the individual. This amount prevents, alleviates, abates, or otherwise reduces the severity of a symptom associated with neoplasia in a patient, such as a reduction in tumor mass or volume or a slowing of tumor growth rate.

The terms “treat,” “treatment” and “therapeutic effect” as used herein refer to reducing or stopping the inflammation process or slowing or halting the release of inflammatory cytokines or reducing the number of neutrofil infiltration.

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “liposome” includes a single liposome as well as two or more of the same or different liposomes, reference to an “excipient” includes a single excipient as well as two or more of the same or different excipients, and the like.

Acute lung injury is defined by a constellation of clinical criteria that involves acute onset of bilateral pulmonary infiltrates with hypoxemia without evidence of hydrostatic pulmonary edema. It has a high incidence and overall mortality remains high. Pathogenesis of lung injury is explained by injury to both the vascular endothelium and alveolar epithelium. Recent advances in the understanding of pathophysiology have identified several biologic markers that are associated with worse clinical outcomes.¹

Acute respiratory distress syndrome is a life-threatening condition characterized by poor oxygenation and non-compliant or “stiff” lungs. The disorder is associated with capillary endothelial injury and diffuse alveolar damage. Once ARDS develops, patients usually have varying degrees of pulmonary artery vasoconstriction and may develop pulmonary hypertension. ARDS carries a high mortality, and few effective therapeutic modalities exist to ameliorate this deadly condition.

Lung inflammation may also result from upper or lower respiratory tract infection arising from bacteria or virus. Lower respiratory tract infections generally are considered to include acute bronchitis, bronchiolitis, influenza, and pneumonia.

Many viruses have characteristic seasonal patterns. Influenza virus and respiratory syncytial virus infections peak in winter, but other respiratory viruses, such as human metapneumovirus (hMPV), parainfluenza viruses, and coronaviruses are also prevalent in the fall and winter. Respiratory viruses include, but are not limited to, adenovirus and rhinovirus, which may cause illness year-round. Respiratory viruses include adenovirus, influenza A, B, C, parainfluenza virus, respiratory syncytial virus, human coronavirus, human metapneumovirus, and the severe acute respiratory syndrome-associated CoVs, SARS-COV-1 and in 2019 SARS-COV-2.

Respiratory tract bacterial infections include the following: Bordetella pertussis, Chlamydophila pneumoniae, Mycoplasma pneumoniae, Streptococcus pneumoniae, Klebsiella pneumoniae, Staphylococcus aureus, Pseudomonas aeruginosa, Escherichia coli, Haemophilus influenza, Legionella pneumophila, and Acinetobacter and Enterobacter species. Two important bacterial lower respiratory tract infections include acute exacerbations of chronic obstructive pulmonary disease (COPD) and community-acquired pneumonia.

Chronic inflammation of COPD is characterized by the accumulation of neutrophils, macrophages, B cells, CD4⁺-T, CD8⁺-T cells, dendritic cells, and eosinophils, particularly in the smaller airways, and the severity of COPD is associated with the infiltration of these inflammatory immune cells. The role of inflammatory cells in COPD has focused on oxidants, proteinases, perforin, and granzymes released from these cells, leading to alveolar wall destruction and mucus hypersecretion.⁴

The generally known pharmaceutical term “liposomes” denotes colloidal particles that form spontaneously when phospholipids are dispersed in an aqueous medium. A particular feature for a medical application of such liposomes is that during the formation of liposomes, phospholipids organize in the form of a membrane, which is very similar to the natural membrane of cells and cell organelles. Simultaneously, a certain fraction of the aqueous solution is encapsulated in the inner compartment of liposomes, which therefore can be used for the delivery of lipophilic, i.e., membrane-bound, and also hydrophilic, i.e., solubilized in the encapsulated aqueous compartment, therapeutic agents.

In order to circumvent the problem of loss of drug when given by oral route, efforts are made to deliver the drug to the target site or the immediate vicinity thereof that bypass the gastrointestinal tract. An inhalation administration is, for example, suitable for the treatment of lung diseases.

A prerequisite for an efficient inhalation therapy is the delivery of aerosol particles into the lung, which, in particular, depends on the diameter and density of the particles utilized. A further issue for an inhalation administration of liposomes is their stability during the nebulization process. During nebulization of suspensions and liquids, liposomes in the aerosol are often subjected to forces that may compromise liposome integrity, thus leading to a premature release of liposome-encapsulated compounds. Inhaled drug compounds often have a short duration of action. As a result, inhalations, in most cases, have to be carried out in short intervals.

The present liposomal composition provides liposomes that will simultaneously deliver two drugs differing in their hydrophilicity and also exhibit a high stability during nebulization. At the same time, aerosols prepared from liposomal formulations should be able to easily reach the lung and provide biologically compatible liposomes, which also allow for a sustained action of the enclosed active substances in the target tissue. Furthermore, the preparation of such liposomes should be convenient, reliable, and cost-effective. Beyond this, the possibility should be provided to prepare pharmaceutical formulations that are suitable for

In one aspect, a composition comprising liposomes that co-encapsulate cysteamine and resveratrol are provided. The components of the liposome composition will now be described.

Cysteamine is a hydrophilic chemical compound having the empirical formula C2H7NS and the structural formula shown as formula I below:

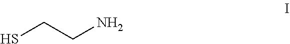

It is well known for treating cystinosis, but in some applications it is used to take advantage of its antioxidant properties, since it is known to increase intracellular glutathione levels.

Resveratrol (3,5,4′-trihydroxy-trans-stilbene) is a lipophilic compound, a polyphenol naturally occurring in some plants, including grapes, blueberries, raspberries, mulberries and peanuts. Resveratrol has the structure shown below in formula II:

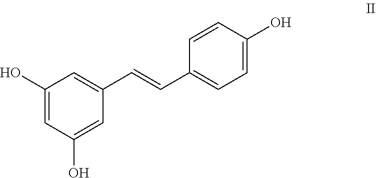 [SMILES: Oc1ccc(/C=C/c2cc(O)cc(O)c2)cc1]

Like other naturally occurring polyphenols, resveratrol is known to exhibit antioxidant properties, and forms part of the plant's defense mechanism when attacked by some pathogens, such as bacteria and fungi.

The liposomal resveratrol provided for use in the methods described herein is, in one embodiment, comprised of resveratrol releasably attached to a lipophilic or hydrophobic moiety.

Liposomes comprising resveratrol and cysteamine were prepared as detailed in Example 1. The exemplary liposomes had an external coating of D-αtocopheryl polyethylene glycol 1000 succinate (TPGS), as described, for example, in research paper by Kotta et al., 2021. The liposomes were prepared using phosphatidyl choline, TPGS, cholesterol and at a percent molar ratio of 55:5:30:10, respectively. The liposomes were incubated with cysteamine to load cysteamine into the aqueous space of the liposomes. The liposomes with co-encapsulated cysteamine and resveratrol were used in most of the studies now to be described.

### Example 1

### Synthesis of Liposomes

Liposome formation was performed using the spontaneous formation method in which all the lipid constituents were melted by heating at 55° C. and resveratrol was dissolved in it. The mixture was hydrated using water with cysteamine dissolved in it and heated to 55° C. After hydration the mixture was passed through a polycarbonate membrane extrusion with 80 nm pore size. The formulation was sterilized using 0.45 μm and 0.22 μm filters. The final formulation of the liposome compositions is shown in Table 1, below.

TABLE 1
Composition of Liposomes

| Final Volume | 30 | mL |
|---|---|---|
| pH | 6 | |
| Osm | 290 | |
| Size | 120 | nm |
| Polydispersity Index | 0.05 | |
| Lipids | 10 | μmol/mL |
| Resveratrol | 2 | μmol/mL |
| Cysteamine | 3.2 | μmol/mL |

### Example 2

### Characterization of Liposomes

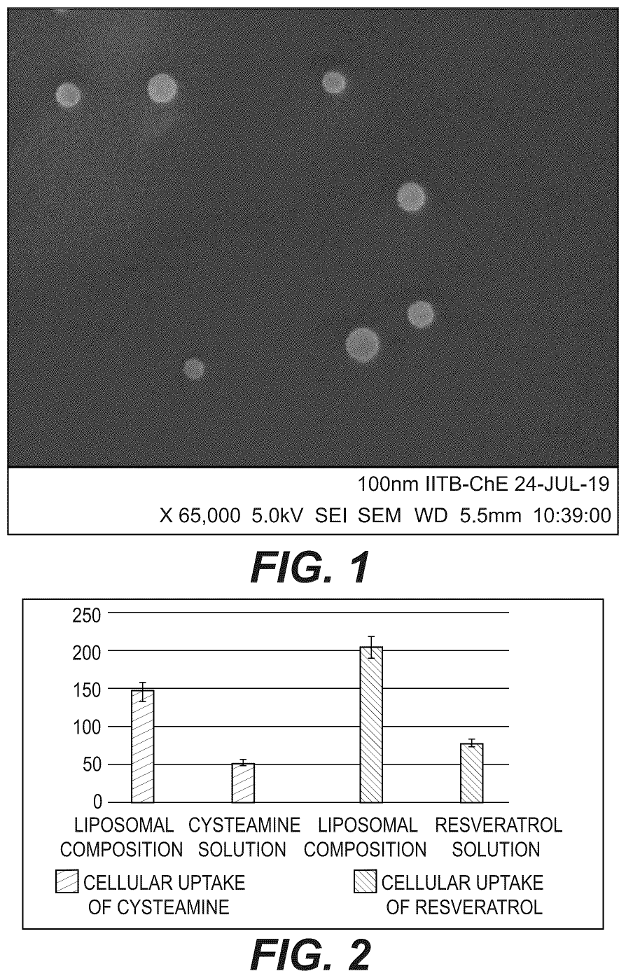

Liposomes prepared according to Example 1 were inspected by electron cryomicroscopy (cryoSEM). An exemplary image is shown in FIG. 1.

### Example 3

### In Vitro Characterization: Cell Uptake and Biocompatibility

The cytotoxicity of liposomes with co-encapsulated cysteamine and resveratrol were evaluated in A549 cells (human lung adenocarcinoma epithelial cells). For comparison pristine resveratrol and pristine cysteamine were also tested. Cell uptake studies were done as follows. 1×10⁶ cells were incubated for 3 hours with the various test agents at a cysteamine concentration of 6.5 μmoles/mL and final resveratrol concentration of 5 μmoles/mL. Results are shown in FIG. 2.

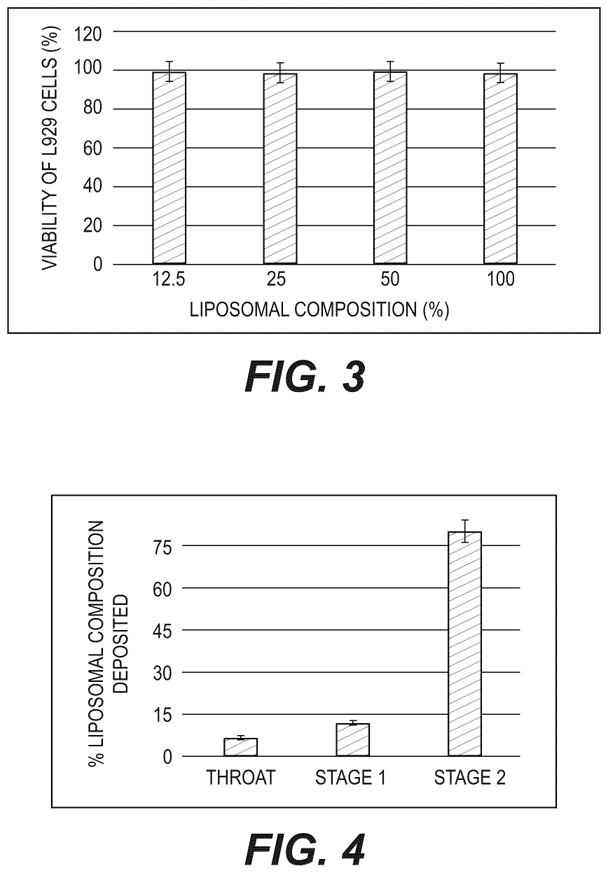

The liposomal composition was evaluated for cell viability in the mouse fibroblast cell line (L929). The cells were maintained in DMEM (Dulbecco's modified Eagle medium) and distributed in 96-well plates (1×10⁴ cells/well) for 24 h in a 5% CO₂ atmosphere at 37° C. After this period, the medium was removed and the adhered cells were treated with formulations (4 dilutions taking 0.2 g/mL of formulation in DMEM as 100%, and further dilution with DMEM to obtain 50%, 25%, and 12.5%, n=6) under the same incubation conditions. Untreated cells were used as controls and considered with 100% cell viability. The viability was determined by MTT assay as described in ISO 10993-5. MTT (1 mg/mL in HBSS) was added to each well of the plate, which was again incubated for 3 h in 5% CO₂ atmosphere at 37° C. After this, the medium was aspirated, and the formazan crystals formed were dissolved in DMSO. After 30 min, the optical density was measured on a microplate reader at the wavelength of 570 nm, and the percentage of viability was expressed as the growth rate (%) in comparison to control. Results are shown in FIG. 3.

### Example 4

### Anderson Cascade Impactor

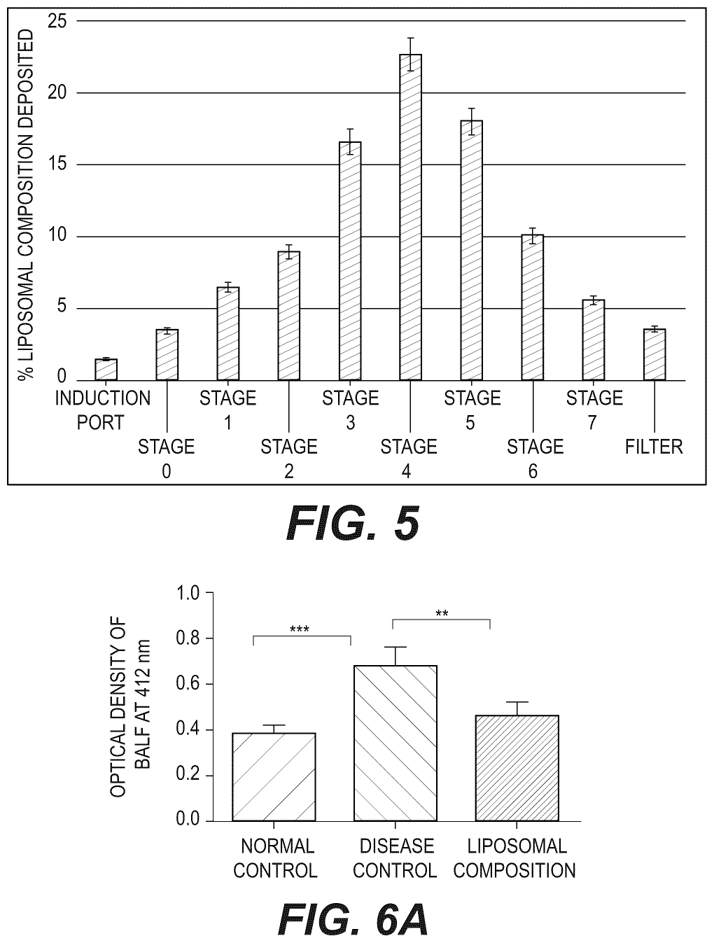

An Anderson Cascade Impactor was used to measure the mass median aerodynamic diameter. Results are shown in FIGS. 4 and 5.

### Example 5

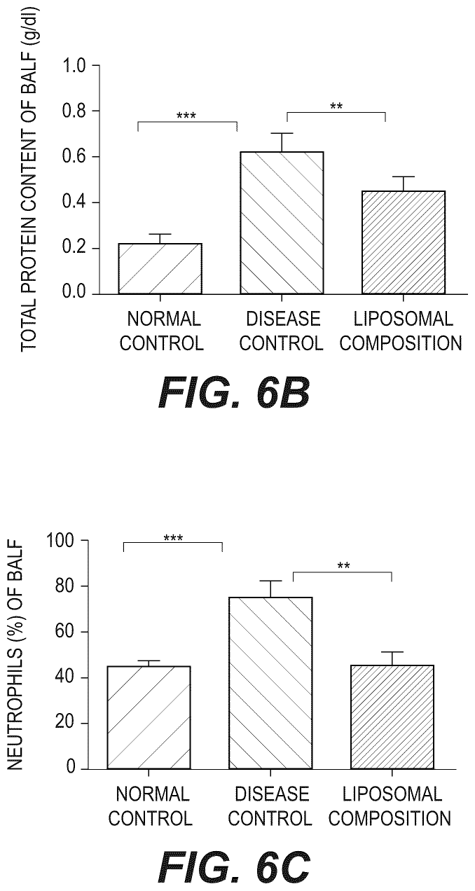

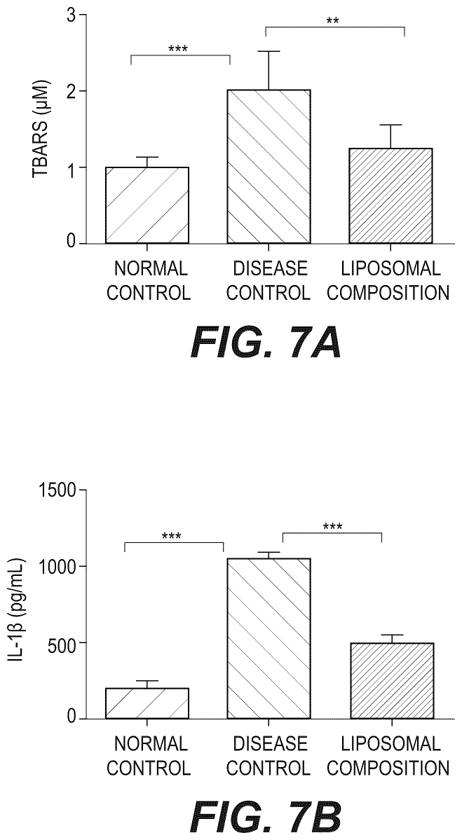

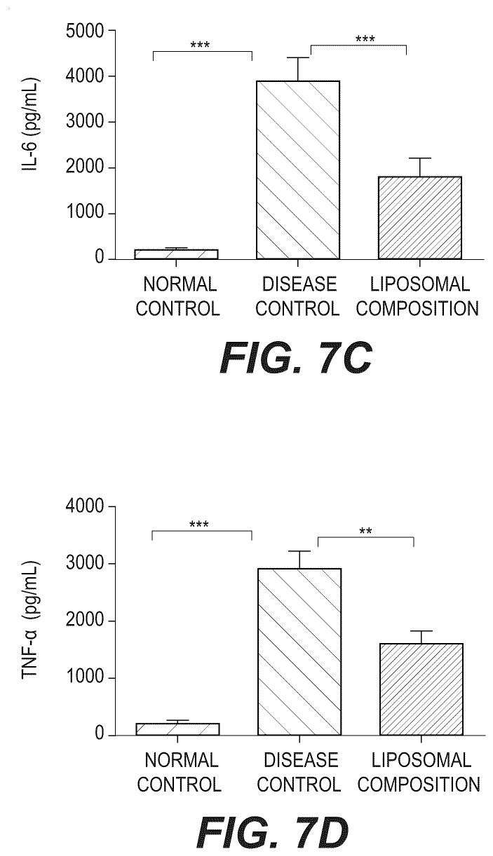

Wistar rats (weight 180-220 g) were divided into four groups, with six rats in each group, namely, normal control, diseased control, and test treatment. All the rats were anesthetized with an intraperitoneal injection of ketamine-xylazine (1:1). Sterile Lipopolysaccharide from Escherichia coli 0111:B4 solution in normal saline (5 mg/kg body weight) was administered intratracheally to all the rats, except the normal control group. Normal control healthy group rats received normal saline. Animals were observed for any adverse symptoms every 30 min from the time of intratracheal LPS instillation until the end of study. Rats were sacrificed 24 h post-dosing and bronchioalveolar lavage fluid (BALF) was collected. The BALF was analyzed for neutrophils (%), total protein, and pulmonary hemorrhage. Results are shown in FIGS. 6A, 6B, and 6C. Levels of lipid peroxides (Thiobarbituric acid reactive substances; TBARS), IL-1β, IL-6, and TNF-α in BALF were analyzed using ELISA kits. Results are shown in FIGS. 7A, 7B, 7C, and 7D.

It should be noted that the present liposomal composition provides an immediate response to lung inflammation by the hydrophilic cysteamine core and a prolonged, sustained response by the lipophilic resveratrol lipid layer. Dosage and frequency of administration may be determined by one of ordinary skill in the art without undue experimentation by routine monitoring of the patient's response to treatment.

It is to be understood that the liposome encapsulating cysteamine and resveratrol in separate compartments is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of treating a patient suffering from an acute lung injury or severe acute respiratory distress syndrome, comprising a step of intratracheally administering to the patient a therapeutically effective amount of a dual drug loaded liposome for reducing inflammation of the lung caused by the acute respiratory distress syndrome or the respiratory distress syndrome; wherein the dual drug loaded liposome comprises a non-pegylated dual drug loaded liposomal composition containing two drugs differing in their hydrophilicity and lipophilicity, comprising:
a lipid outer shell;
a hydrophilic inner core disposed within the outer shell;
a hydrophilic synthetic antioxidant that is one of the two drugs disposed within the hydrophilic inner core; and
a lipophilic natural antioxidant that is the other of the two drugs disposed within a lipid layer between the inner core and the outer shell,
wherein the non-pegylated dual drug loaded liposomal composition is configured for pulmonary administration to a patient in need thereof.

2. The method according to claim 1, wherein the hydrophilic synthetic antioxidant comprises cysteamine or a pharmaceutically acceptable salt thereof which is encapsulated within the hydrophilic inner core.

3. The method according to claim 1, wherein the lipophilic natural antioxidant comprises resveratrol or a pharmaceutically acceptable salt thereof which is encapsulated inside the outer shell.

4. A method of treating a patient suffering from an acute lung injury or respiratory distress syndrome, comprising a step of intratracheally administering to the patient a therapeutically effective amount of a dual drug loaded liposome for reducing inflammation of the lung caused by the acute lung injury or the respiratory distress syndrome, wherein the dual drug loaded liposome comprises a dual drug loaded liposome encapsulating cysteamine and resveratrol having differing hydrophilicity in separate compartments, comprising a liposome having an inner core, an outer shell encapsulating the inner core, and a lipid layer disposed between the inner core and the outer shell, the inner core being hydrophilic and including cysteamine, the lipid layer being lipophilic and including resveratrol, wherein the dual drug loaded liposome composition is configured for pulmonary application suitable for intratracheal administration to a patient in need thereof.

5.-10. (canceled)

11. A method of preparing the non-pegylated dual-drug loaded liposomal composition as claimed in claim 1, the method comprising:
a. forming a liposome using a spontaneous formation method in which all lipid constituents are melted by heating at 55° C. and dissolving resveratrol therein;
b. hydrating the liposome using water with cysteamine dissolved therein and heating to 55° C.;
c. passing the liposome through a polycarbonate membrane extrusion with 80 nm pore size;
d. sterilizing the liposome using 0.45 μm and 0.22 μm filters; and
e. obtaining the non-pegylated dual-drug loaded liposomal composition.

12. The method according to claim 1, wherein the liposomal composition has a particle size of 120 nm.

13. The method according to claim 4, wherein the liposome has a particle size of 120 nm.

14. The method according to claim 1, wherein the non-pegylated dual drug loaded liposomal composition has a mass median aerodynamic diameter measurement using Anderson Cascade Impactor showing more than 21% deposition on stage 4.

15. The method according to claim 4, wherein dual drug loaded liposome has a mass median aerodynamic diameter measurement using Anderson Cascade Impactor showing more than 21% deposition on stage 4.
